(19) United States
(12) Patent Application Publication
(10) Pub. No.: US 2006/0235263 A1
(43) Pub. Date: Oct. 19, 2006

(54) Palpable, implantable actuator

(75) Inventors: Andrew Jacobson, San Antonio, TX (US); William Paff, San Antonio, TX (US)

Correspondence Address: SKOKOS LAW GROUP, LLC, 1100 JORIE BOULEVARD, SUITE 220, OAK BROOK, IL, 60523, US

(73) Assignee: Jacobson Technologies, LLC

(21) Appl. No.: 11/378,083

(22) Filed: Mar. 17, 2006

Related U.S. Application Data
(60) Provisional application No. 60/663,172, filed on Mar. 18, 2005.

(51) Int. Cl. A61F 2/02

(52) U.S. Cl. 600/31

(57) ABSTRACT

A palpable, implantable actuator that may be used to remotely actuate a mechanism, component or device that has been implanted completely within an animal's or human's body and is located distally from the palpable, implantable actuator. The palpable, implantable actuator is implanted completely within the body and is palpable and able to be manipulated through the skin of the body.

### CROSS REFERENCE TO RELATED APPLICATION

The present application is a regular utility application claiming the benefits of the filing date of provisional application, Application No. 60/663,172, filed on Mar. 18, 2005 by the present applicant.

### BACKGROUND OF THE INVENTION

The present invention relates generally to an implantable medical device and more particularly to a palpable, implantable actuator that may be used to remotely actuate a mechanism, component or device that has been implanted completely within and is operated entirely within an animal's or human's body.

While certain mechanical actuators currently exist that allow a user to remotely actuate a mechanism, component or device that is located distally from the actuator, such medical devices are not implanted completely within an animal's or humans' body. Such partially exteriorized mechanical actuators include various surgical devices such as laparoscopic instruments and various wire-based catheter devices used for interventional procedures. Similarly, completely implanted mechanical devices exist which allow a user to actuate a mechanism or component located distally from the actuator. Examples of such completely implanted devices include an inflatable polymeric gastric banding device connected to an implanted port reservoir for obesity treatment in humans and an inflatable polymeric vascular occluder connected to an implanted port reservoir in laboratory animals. These medical devices, however, require transcutaneous access by a non-implanted needle to supply fluid or pneumatic pressure to effect actuation.

The advantages of a completely implanted device that does not require transcutaneous access by a needle for actuation include a reduced rate of infection when compared with a device which is both partially implanted and partially exteriorized or a completely implanted device which requires transcutaneous needle access to activate. A device that is implanted completely within the body that does not require transcutaneous access by a needle for actuation is not exposed to the potentially infectious microorganisms that are present on the skin of a human or animal thereby reducing the risk of device-related infection. In addition to the reduced rate of infection, devices that are completely implanted within the body obviate the need for dressings that would be required for devices that are partially exteriorized from the body. A human with a completely implanted device, therefore, is able to engage in activities that are problematic with a partially implanted and partially exteriorized device including showering, swimming, or any type of activity that increases the likelihood of infection or place stress on the entrance point of a partially exteriorized and partially implanted device into the body. Also, the use of a completely implanted actuator removes some of the stigma associated with illness by allowing a human to resume everyday activities and by improving cosmesis. In addition, by having such an actuator fully implanted within the body, there is no need for external securement devices thereby adding to the comfort of a human and animal. Finally, by having the actuator fully implanted within the body, there is a reduced risk of damage to the device and to the human's or animal's health.

### BRIEF SUMMARY OF THE INVENTION

The present invention is directed to a palpable, implantable actuator comprising a mechanical switch that is connected to activation means that may be used to remotely actuate a mechanism, component or device that has been implanted completely within an animal's or human's body and is located distally from the palpable, implantable actuator. The palpable, implantable actuator is implanted completely within the body but is palpable and able to be manipulated through the skin of the body in order to activate the mechanical switch. A connector may connect the activation means to the mechanical switch. The connector may be but is not limited to polymeric or metallic linear wire, polymeric or metallic curvilinear wire, pneumatic tubing, hydraulic tubing, rod, cylinder, wire bundle, string, cord, or spring. The activation means of the palpable, implantable actuator may be but are not limited to a clamp, valve, cutting tool, conductive element, switch, piston, suture, manipulator, balloon, fabric, membrane, hook, stirrup, wire, noose, or any other element, component, or device which is activated to actuate surrounding bodily tissue, to intervene in or monitor the body's activities, or to actuate an implanted medical device which will in turn actuate surrounding bodily tissue or intervene in or monitor the body's activities.

The mechanical switch may have multiple positions including but not limited to “deactivated” and “activated” positions, as is the case with a two-position switch, and multiple intermediate positions that vary between the “deactivated” position and “activated” position. In the “deactivated” position, the mechanical switch of the palpable, implantable actuator may be in the position whereby the connector is withdrawn toward the mechanical switch resulting in the activation means being in the “deactivated” position, located proximally to the mechanical switch. To cause the switch to be in the “activated” position, the mechanical switch has been palpated and manipulated through the skin by the user such that the switch causes the connector to be fully extended causing the activation means to be in the “activated” position, located distally from the mechanical switch. The mechanical switch of the palpable, implantable actuator may be inverted wherein the “deactivated” and “activated” positions are reversed. In a palpable, implantable actuator where the mechanical switch is inverted and the “deactivated” and “activated” positions are reversed, the withdrawn position of the connector may correspond to the “activated” position wherein the activation means are “activated,” while the fully extended position of the connector may correspond to the “deactivated” position wherein the activation means are “deactivated.”

### BRIEF DESCRIPTION OF THE DRAWINGS

FIG. 1a is side cross sectional view of the palpable, implantable actuator designed in accordance with an embodiment of the present invention, the figure illustrating a two position mechanical switch in the “deactivated” position;

FIG. 1b is side cross sectional view of the palpable, implantable actuator designed in accordance with an embodiment of the present invention, the figure illustrating a two position mechanical switch in an “activated” position;

FIG. 2a is side cross sectional view of the palpable, implantable actuator designed in accordance with an embodiment of the present invention, the figure illustrating a two position mechanical switch in the “deactivated” position;

FIG. 2b is side cross sectional view of the palpable, implantable actuator designed in accordance with an embodiment of the present invention, the figure illustrating a two position mechanical switch in an “activated” position;

FIG. 3a is side cross sectional view of the palpable, implantable actuator designed in accordance with an embodiment of the present invention, the figure illustrating a multi-position mechanical switch in the “deactivated” position;

FIG. 3b is side cross sectional view of the palpable, implantable actuator designed in accordance with an embodiment of the present invention, the figure illustrating a multi-position mechanical switch in an “intermediate” or partially “activated” position;

FIG. 3c is side cross sectional view of the palpable, implantable actuator designed in accordance with an embodiment of the present invention, the figure illustrating a multi-position mechanical switch in an “activated” position;

FIG. 4a is side cross sectional view of the palpable, implantable actuator designed in accordance with an embodiment of the present invention, the figure illustrating a multi-position mechanical switch in the “deactivated” position;

FIG. 4b is side cross sectional view of the palpable, implantable actuator designed in accordance with an embodiment of the present invention, the figure illustrating a multi-position mechanical switch in an “intermediate” or partially “activated” position;

FIG. 4c is side cross sectional view of the palpable, implantable actuator designed in accordance with an embodiment of the present invention, the figure illustrating a multi-position mechanical switch in an “activated” position;

FIG. 5a is side cross sectional view of the palpable, implantable actuator designed in accordance with another embodiment of the present invention, the figure illustrating a piston 29 within a fluid or pneumatic tube 31 as the connector and the two position mechanical switch in a “deactivated” position;

FIG. 5b is side cross sectional view of the palpable, implantable actuator designed in accordance with another embodiment of the present invention, the figure illustrating a piston 29 within a fluid or pneumatic tube 31 as the connector and the two position mechanical switch in an “activated” position;

FIG. 6a is side cross sectional view of the palpable, implantable actuator designed in accordance with another embodiment of the present invention, the figure illustrating a piston 29 as the connector within a fluid or pneumatic tube 31 and the two position mechanical switch in a “deactivated” position;

FIG. 6b is side cross sectional view of the palpable, implantable actuator designed in accordance with another embodiment of the present invention, the figure illustrating a piston as the connector 29 within a fluid or pneumatic tube 31 and the two position mechanical switch in an “activated” position;

FIG. 7 is side cross sectional view of a system for total bile collection comprising the palpable, implantable actuator designed in accordance with an embodiment of the present invention, a bile tube 33, and a sampling tube 35, the figure illustrating the user palpating the mechanical switch 3 and applying pressure to change from a “deactivate” position to an “activated” position to cause the activation means to occlude the bile tube 33;

FIG. 8 is side cross sectional view of a system for total bile collection comprising the palpable, implantable actuator designed in accordance with an embodiment of the present invention, a bile tube 33, and a sampling tube 35, the figure illustrating the palpable, implantable actuator and activation means in an “activated” position thereby causing the activation means to occlude the bile tube 33 and diverting bile to flow into the sampling tube 35;

FIG. 9 is perspective view of the palpable, implantable actuator designed in accordance with another embodiment of the present invention wherein the mechanical switch is spring loaded to prevent inadvertent “activation” of the mechanical switch, the figure illustrating the connector 7 fully extended;

FIG. 10 is perspective view of the palpable, implantable actuator designed in accordance with another embodiment of the present invention wherein the mechanical switch is spring loaded to prevent inadvertent “activation” of the mechanical switch, the figure illustrating the connector 7 fully withdrawn;

FIG. 11 is perspective view of the palpable, implantable actuator designed in accordance with another embodiment of the present invention wherein the mechanical switch is spring loaded to prevent inadvertent “activation” of the mechanical switch, the figure illustrating the connector 7 fully withdrawn; and

FIG. 12 is perspective view of the palpable, implantable actuator designed in accordance with another embodiment of the present invention wherein the mechanical switch is spring loaded to prevent inadvertent “activation” of the mechanical switch, the figure illustrating the connector 7 fully extended.

### DETAILED DESCRIPTION OF THE DRAWINGS

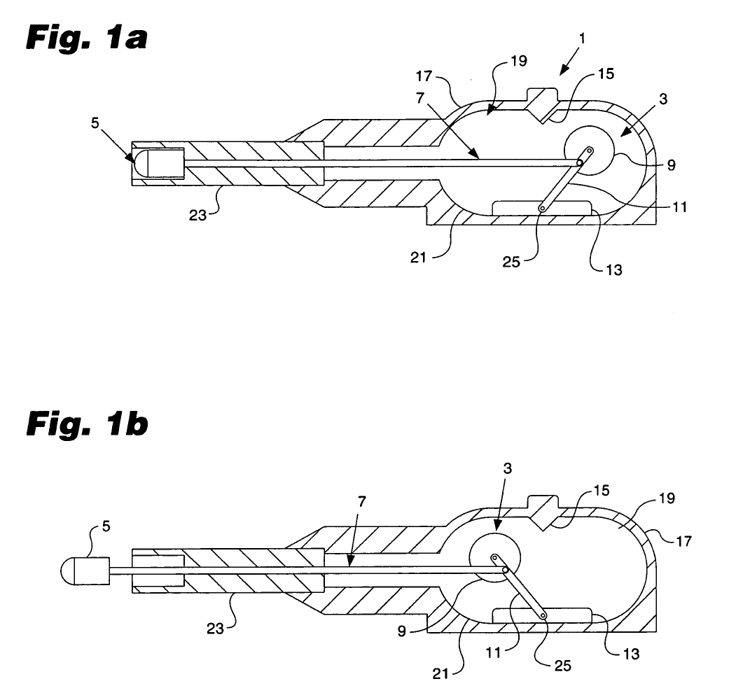

FIGS. 1(a) and (b) illustrate an embodiment of the palpable, implantable actuator, shown generally at 1. The palpable, implantable actuator 1 comprises a mechanical switch 3 that is connected to activation means 5 that may be used to remotely actuate surrounding bodily tissue or a mechanism, component or device that has been implanted completely within an animal's or human's body and is located distally from the palpable, implantable actuator 1. In this embodiment, the mechanical switch 3 may be enclosed in a flexible shell 17 that allows a user to manually activate the palpable, implantable actuator by applying finger pressure through the skin. Alternatively, the user may use an external device to apply pressure through the skin. The flexible shell 17 may have an interior cavity containing at least one catch or ridge 15 in the center of the top portion of the interior wall 19 of the cavity thereby allowing the mechanical switch to be secured and palpated in an “activated” or “deactivated” position.

In this embodiment, the connector 7 may be a polymeric or metallic linear, or curvilinear, wire connecting the mechanical switch 3 at one end of the connector 7 to the activation means 5 at the opposite end of the connector 7. The implantable, palpable actuator may have a sheath 23 that is concentric to the connector 7 and provides a protective barrier and passageway for the connector 7 to pass. The sheath 23 also adds to the functioning of the connector 7 by reinforcing the connector 7 as the mechanical switch is “activated” and the connector 7 becomes fully extended.

The activation means 5 of the palpable, implantable actuator may be but are not limited to a clamp, valve, cutting tool, conductive element, switch, piston, suture, manipulator, balloon, fabric, membrane, hook, stirrup, wire, noose, or any other element, component, or device which is activated to actuate surrounding bodily tissue, to intervene in or monitor the body's activities, or to actuate an implanted medical device which will in turn actuate surrounding bodily tissue or intervene in or monitor the body's activities. In FIGS. 1(a) and (b), the activation means 5 are shown as a plug used to occlude the passage of fluid or other material through a biological or synthetic passageway or organ or to apply force to a part of the body.

To secure the mechanical switch to the “activated” position, the user applies finger pressure, or uses an external device, to push the mechanical switch 3 in one direction within the flexible shell 17 toward the connector 7, thereby causing the connector 7 to extend axially through the sheath 23 and allow the activation means 5 to intervene in or monitor the body's activities or to remotely actuate a medical device that also has been implanted beneath the skin and is located a distance from the palpable, implantable actuator 1. The palpable, implantable actuator 1 may be deactivated by applying finger pressure, or use an external device to apply pressure, through the skin to push the mechanical switch 3 in the opposite direction thereby returning the mechanical switch 3 to its “deactivated” position and allowing the connector 7 and activation means 5 to retract to their original “deactivated” position. An advantage of this invention is that while the user of the palpable, implantable actuator 1 is “blind” when palpating the mechanical switch 3 due to the fact that the palpable, implantable actuator 1 is completely implanted within a human's or animal's body, the palpable, implantable actuator 1 provides tactile feedback for the user to discern whether the palpable, implantable actuator 1 has been activated or deactivated. An additional advantage of this embodiment of the invention over current devices which are hydraulically or pneumatically actuated via a transcutaneous needle is the reduced risk of complications. In a two position mechanical switch, as shown in FIGS. 1(a)-(b), there is no risk of providing too much or too little fluid or air pressure for the actuation as with implanted devices which are hydraulically or pneumatically actuated via a transcutaneous needle. In the present embodiment of the invention, the user simply applies force to the mechanical switch 3 and since the sheath 23 provides tactile feedback to the user, the user immediately is aware if the user has provided sufficient force to “activate” or “deactivate” the palpable, implantable actuator 1.

The mechanical switch 3 may be reversible, irreversible or adjustable and may include numerous states of activation including “activated” and “deactivated” as well as multiple “intermediate” positions where the mechanical switch is partially activated in positions between the “activated” and “deactivated” positions.

The flexible shell 17 may be of a biocompatible flexible polymer that allows the user to push the mechanical switch 3 through the flexible shell 17 into the “activated” or “deactivated” position. The mechanical switch 3 may be of a polymer or metal that is biocompatible and acceptable for subcutaneous medical devices. The sheath 23 may also be of a biocompatible polymer or metal to provide the connector 7 with protection and a passageway. The connector 7 may be of a polymer or metal that is biocompatible and acceptable for subcutaneous medical devices.

In one embodiment of the invention, as shown in FIGS. 1(a),(b) and 3(a),(b) and (c), the mechanical switch 3 may be a toggle switch comprising a flexible shell 17, a base 13 that is fixed to the bottom portion of the interior wall 21 of the cavity of the flexible shell 17, and a toggle switch comprising a shaft 11 of which one end is connected to the base 13 via a pin 25 and is allowed rotate relative to the pin 25, and a palpable portion 9 that may be a shaped object that is easily palpable and that is fixed to the other end of the shaft 11. The connector 7 may be attached to the shaft 11 of the toggle switch at any point along the length of the shaft 11. The travel length of the connector 7 is allowed to be varied depending on the location of the attachment of the connector 7 to the shaft 11 of the toggle switch. By attaching the connector 7 higher on the shaft 11 relative to the base 13 of the toggle switch, the connector 7 will travel a greater distance than when the connector 7 is attached lower on the shaft 11 of the toggle switch.

In this embodiment, the flexible shell 17 may have at least one ridge or catch 15 at the top portion of the interior cavity 19 of the flexible shell 17 to secure the shaft 11 and palpable portion 9 of the mechanical switch in the different positions including the “deactivated” and “activated” positions. As shown in FIGS. 1(a) and (b), the mechanical switch may be a two position mechanical switch allowing for “deactivated” and “activated” positions. In the “deactivated” position, the toggle switch is located in the portion of the cavity of the flexible shell 17 most distal from the opening in the flexible shell. In the “deactivated” position, as shown in FIG. 1(a), the connector 7 and activation means 5are located most proximal to the mechanical switch 3. As the user applies force to the palpable portion 9 of the mechanical switch through the flexible shell 17, in the direction of the activation means 5, the shaft 11 rotates forward relative to the pin 25 towards the opening in the flexible shell thereby causing the connector 7 and activation means 5 to extend away from the flexible shell 17 and into the “activated” position.

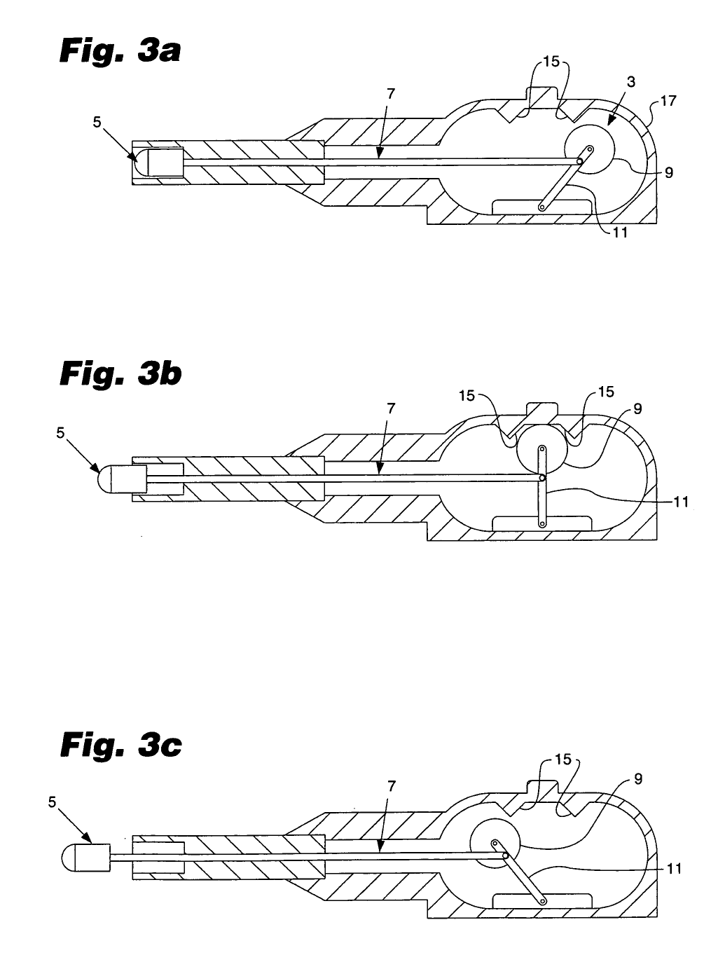

Alternatively, as shown in FIGS. 3(a),(b) and (c), the mechanical switch may be multi-positional allowing for “deactivated,” “intermediate” and “activated” positions. In this embodiment, the flexible shell 17 may have at least two ridges or catches 15 at the top portion of the interior cavity 19 of the flexible shell to secure the shaft 11 and palpable portion 9 of the mechanical switch in the different positions including the “deactivated,” “intermediate” and “activated” positions.

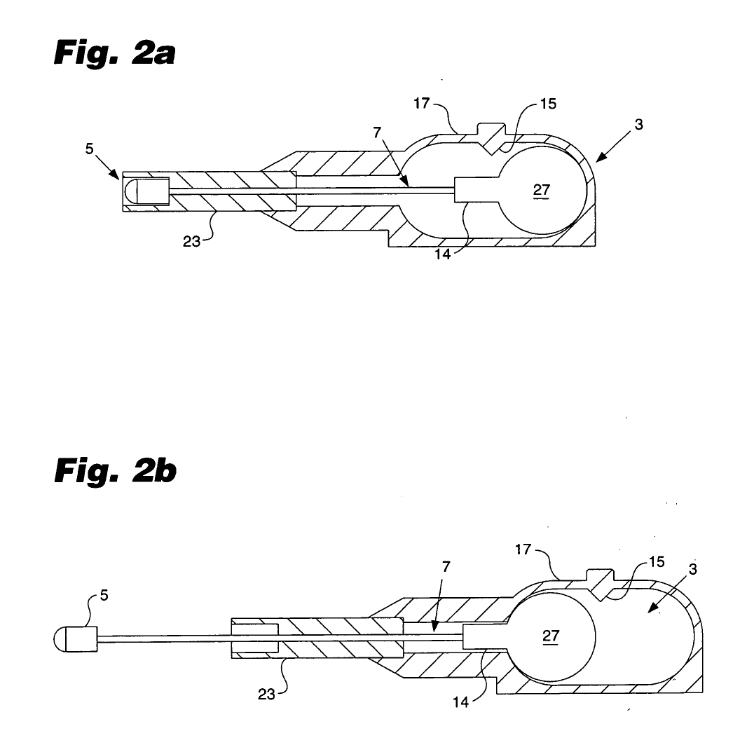

FIGS. 2(a) and (b) illustrate another embodiment of the palpable, implantable actuator. In this embodiment, the mechanical switch 3 comprises a flexible shell 17, a shaft 14 that extends through the interior of the flexible shell 17, and a palpable portion 27 that may be a shaped object that is easily palpable and that is fixed to the end of the shaft 14 opposite of the connector 7. The shaft 14 is in line with the connector 7 and is connected to the connector 7 at one end and to the palpable portion 27 at the opposite end. In this embodiment, the flexible shell 17 may have at least one ridge or catch 15 at the top portion and at least one ridge or catch at the bottom portion of the interior cavity of the flexible shell to secure the shaft 14 and palpable portion 27 of the mechanical switch 3 in the different positions including the “deactivated” and “activated” positions.

As shown in FIGS. 2(a) and (b), the mechanical switch 3 may be a two position mechanical switch allowing for “deactivated” and “activated” positions. In the “deactivated” position, the palpable portion 27 of the mechanical switch is located in the portion of the cavity of the flexible shell 17 most distal from the opening in the flexible shell. In the “deactivated” position, as shown in FIG. 2(a), the connector 7 and activation means 5 are located most proximal to the mechanical switch 3. As the user applies force to the palpable portion 27 of the switch through the flexible shell 17, the palpable portion 27 and shaft 14 are pushed towards the opening in the flexible shell thereby causing the connector 7 and activation means 5 to extend away from the flexible shell 17.

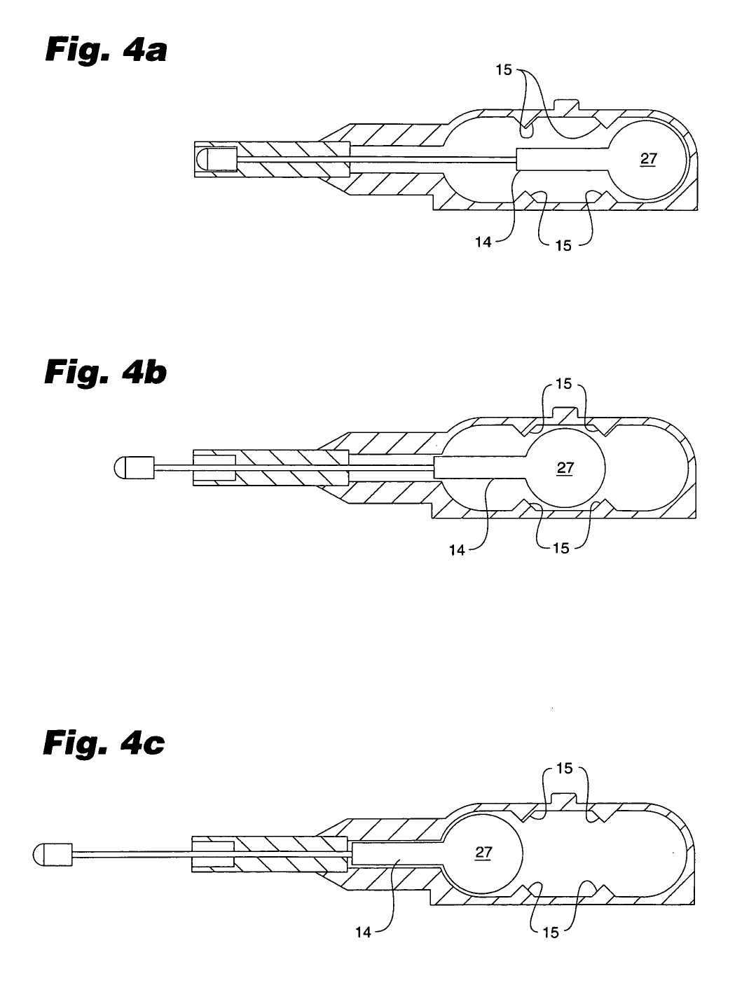

Alternatively, as shown in FIGS. 4(a) (b) and (c), the mechanical switch 3 may be multi-positional allowing for “deactivated,” “intermediate” and “activated” positions. In this embodiment, the flexible shell 17 may have at least two ridges or catches 15 at the top portion of the interior cavity and at least two ridges or catches 15 at the bottom portion of the interior cavity of the flexible shell 17 to secure the shaft 14 and palpable portion 27 of the mechanical switch in the different positions including the “deactivated,” “intermediate” and “activated” positions.

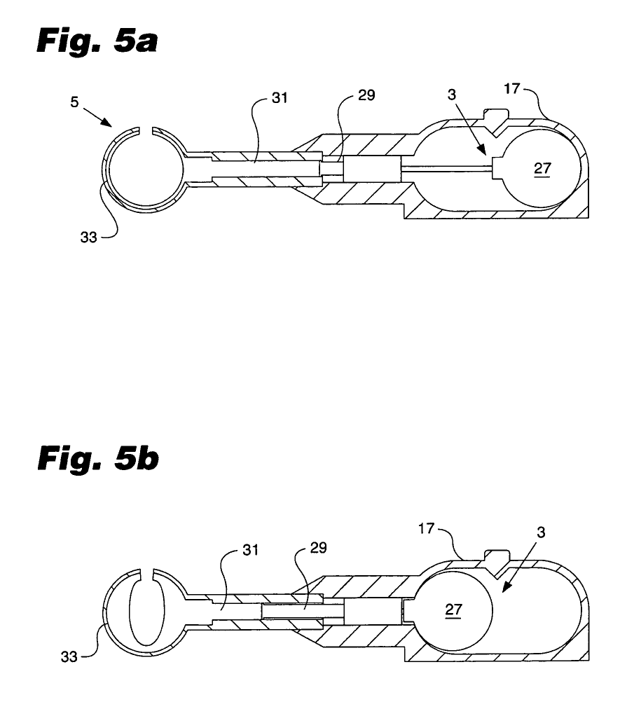

FIGS. 5(a)-(b) and 6(a)-(b) illustrate another embodiment of the palpable, implantable actuator. In this embodiment, the connector is a piston 29 that is connected to the mechanical switch 3. Preferably, the piston 29 is connected at one end to the mechanical switch 3 and at the opposite end is contained within a piston housing 31 that is attached to the activation means 5. As the piston 29 advances forward within the piston housing 31, the air or fluid within the piston housing 31 presses forward thereby causing the activation means 5 to be activated. Alternatively, the connector may be a piston within a piston housing connected at one end to the mechanical switch and at the opposite end lead to another piston connected to the activation means. In the alternative, the connector may be a polymeric or metallic linear wire, polymeric or metallic curvilinear wire, pneumatic tubing, hydraulic tubing, rod, cylinder, wire bundle, cord, or spring connecting the mechanical switch at one end of the connector and connecting to a piston within a piston housing at the opposite end wherein the piston within the piston housing would be connected to the activation means.

FIGS. 5(a)-(b) illustrate one embodiment of the present invention wherein the piston 29 is connected at one end to the palpable portion 27 of the mechanical switch. In the “deactivated” position, as shown in FIG. 5(a), the piston 29 is located most distal to the activation means 5. As the user applies force to the palpable portion 27 of the mechanical switch through the flexible shell 17 in the direction of the activation means 5, the palpable portion 27 of the mechanical switch travels forward towards the opening in the flexible shell thereby causing the piston 29 to advance forward in the piston housing 31 towards the activation means 5. As the piston 29 advances forward, the air or fluid within the piston housing 31 presses forward thereby causing the activation means 5 to be activated. In FIGS. 5(a)-(b), the activation means 5 are shown as a balloon that may occlude a vessel, catheter, or any type of passageway when activated by being inflated. As the piston 29 advances forward, the air or fluid in the piston housing 31 is pressed forward thereby causing the balloon to inflate and consequently occlude the vessel, catheter, or passageway.

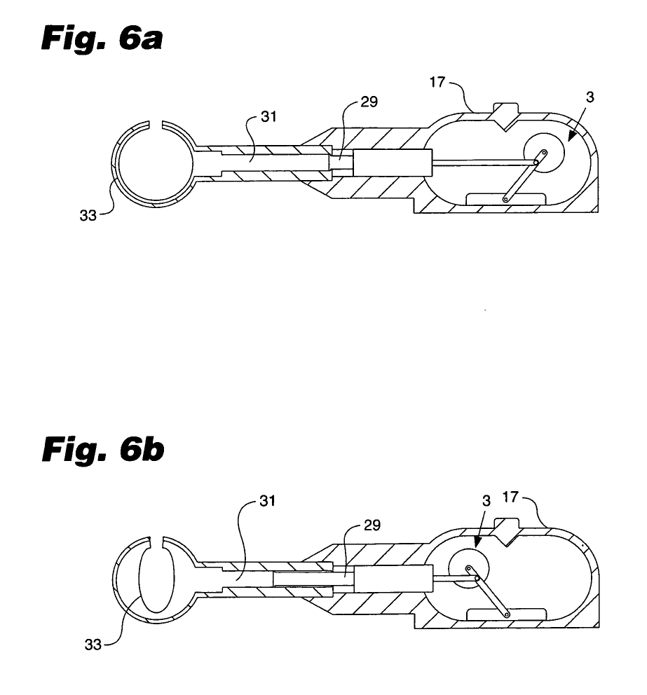

FIGS. 6(a)-(b) illustrate another embodiment of the present invention wherein the piston 29 is connected at one end to the shaft 11 of the toggle switch. In the “deactivated” position, as shown in FIG. 6(a), the piston 29 is located most distal to the activation means 5. As the user applies force to the palpable portion 9 of the mechanical switch through the flexible shell 17 in the direction of the activation means 5, the shaft 11 rotates forward relative to the pin towards the opening in the flexible shell thereby causing the piston 29 to advance forward in the piston housing 31. As the piston 29 advances forward, the air or fluid within the piston housing 31 presses forward thereby causing the activation means 5 to be activated. As in FIGS. 5(a)-(b), the activation means 5 in FIGS. 6(a)-(b) are shown as a balloon that may occlude a vessel, catheter, or any type of passageway when activated by being inflated. As the piston 29 advances forward, the air or fluid in the piston housing 31 is pressed forward thereby causing the balloon to inflate and consequently occlude the vessel, catheter, or passageway.

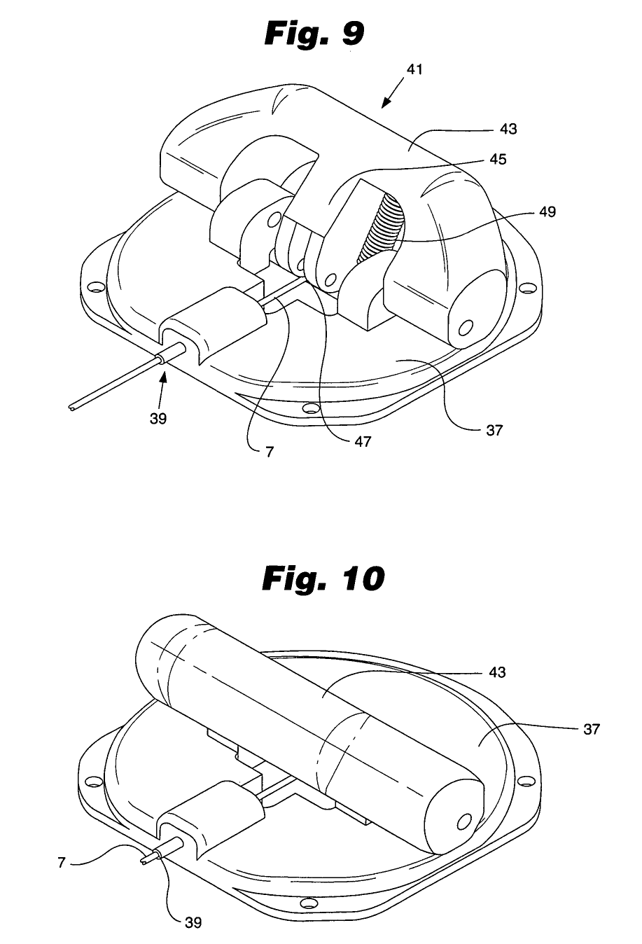

FIGS. 9 and 10 illustrate another embodiment of the palpable, implantable actuator. In this embodiment, the mechanical switch which comprises a base 37 that has one opening 39 in the top surface of the base through which the connector 7 may pass and a spring loaded toggle switch 41 which is connected to the base 37 through pins that allow the toggle switch 41 to rotate relative to the base 37. The toggle switch 41 of this embodiment comprises a top portion 43 and a center projection 45 extending downward from the top portion 43. The connector 7 is attached to the bottom side 47 of the center projection 45 of the toggle switch 41.

In the “deactivated” position, as shown in FIG. 10, the top portion 43 of the toggle switch is located most proximal to the opening in the base 39 through which the connector 7 passes. To cause the toggle switch 41 to move into the “activated” position, as shown in FIG. 9, the user pushes the top portion 43 of the toggle switch away from the opening in the base 39 through which the connector 7 passes. By pushing the top portion 43 of the toggle switch away from the opening in the base 39 through which the connector 7 passes, the bottom of the center projection 47 rotates toward the opening in the base 39 through which the connector 7 passes thereby causing the connector 7 to extend forward away from the base 37. To cause the toggle switch to move into the “deactivated” position, the user pushes the top portion 43 of the toggle switch toward the opening in the base 39 through which the connector 7 passes. By pushing the top portion 43 of the toggle switch toward the opening in the base 39 through which the connector 7 passes, the bottom of the center projection 47 rotates away from the opening in the base 39 through which the connector 7 passes thereby causing the connector 7 to be pulled back towards the base 37.

In this embodiment, at least one spring 49 may be attached at one end to the base 37 and at the opposite end to the top portion 43 of the toggle switch to provide a locking mechanism. The at least one spring 49 secures the toggle switch in the position selected by the user, “activated” or “deactivated,” and prevents inadvertent changing of positions. When the user wants to change the position of the toggle switch, the user must exert a force great enough to overcome the tensile force created by the at least one spring 49 on the toggle switch. The advantage of this invention is the ability to secure the position of the toggle switch without needing the user to separately engage a locking mechanism.

In this embodiment, the mechanical switch may have a flexible shell in order to prevent tissue and debris from gathering in and around the openings, grooves and gaps in the mechanical switch.

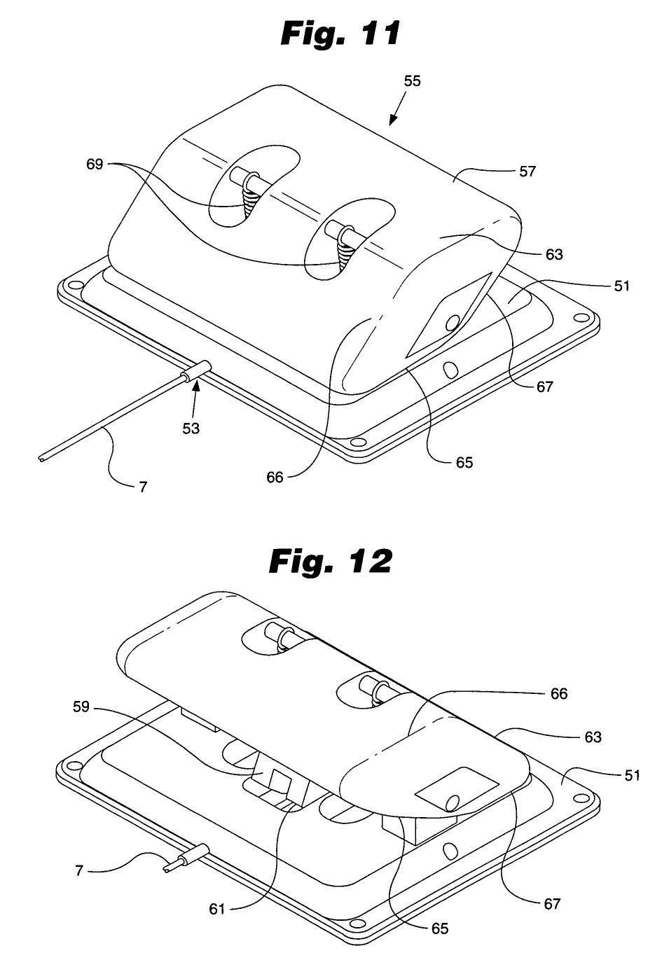

FIGS. 11 and 12 illustrate another embodiment of the palpable, implantable actuator. In this embodiment, the mechanical switch comprises a base 51 that has one opening 53 in the top surface of the base through which the connector 7 may pass and a spring loaded rocker switch 55 which is connected to the base through pins that allow the rocker switch 55 to rotate relative to the base 51. The rocker switch 55 of this embodiment comprises a top portion 57 and a center projection 59 extending downward from the top portion 57. The connector 7 is attached to the bottom side 61 of the center projection 59 of the rocker switch. Preferably, the top portion 57 of the rocker switch may have a rhomboid shape in profile to provide the user with a flat edge to push the rocker switch 55 into the “activated” or “deactivated” position. Alternatively, the rocker switch may be triangular in shape, quarter spherical, or any shape in profile where the angle between a bottom wall and one side wall is greater than ninety degrees and less than one hundred eighty degrees such that the user may depress the top of the rocker switch and cause the rocker switch to move into the “activated” or “deactivated” position.

In the “deactivated” position, as shown in FIG. 11, the top wall 63 and bottom wall 65 of the top portion 57 of the rocker switch 55 is parallel to the base 51 and the bottom wall 65 is pressed against the top surface of the base 51. To cause the rocker switch 55 to move into the “activated” position, as shown in FIG. 12, the user depresses the top wall 63 of the top portion of the rocker switch down such that both side walls 66 and 67 of the rocker switch become parallel to the base 51 and the side wall most distal 67 to the opening 53 in the base becomes pressed against the top surface of the base 51. Also, by pushing the top wall 63 of the top portion 57 of the rocker switch down, the center projection 59 rotates toward the opening 53 in the base through which the connector 7 passes thereby causing the connector 7 to extend away from the base 51. To cause the rocker switch 55 to move into the “deactivated” position, the user depresses the side wall most proximal 66 to the opening 53 in the base such that the top wall 63 and bottom wall 65 become parallel to the base 51 and the bottom wall 65 becomes pressed against the top surface of the base 5 1. By depressing the side wall most proximal 66 to the opening 53 in the base, the bottom 61 of the center projection rotates away from the opening 53 in the base thereby causing the connector 7 to be pulled back towards the base 51.

In this embodiment, at least one spring 69 may be attached at one end to the base 51 and at the opposite end to the top portion 57 of the rocker switch to provide a locking mechanism. As described above, the at least one spring 69 secures the rocker switch in the position selected by the user, “activated” or deactivated,” and prevents inadvertent changing of positions.

In this embodiment, the mechanical switch may have a flexible shell in order to prevent tissue and debris from gathering in and around the openings, grooves and gaps in the mechanical switch.

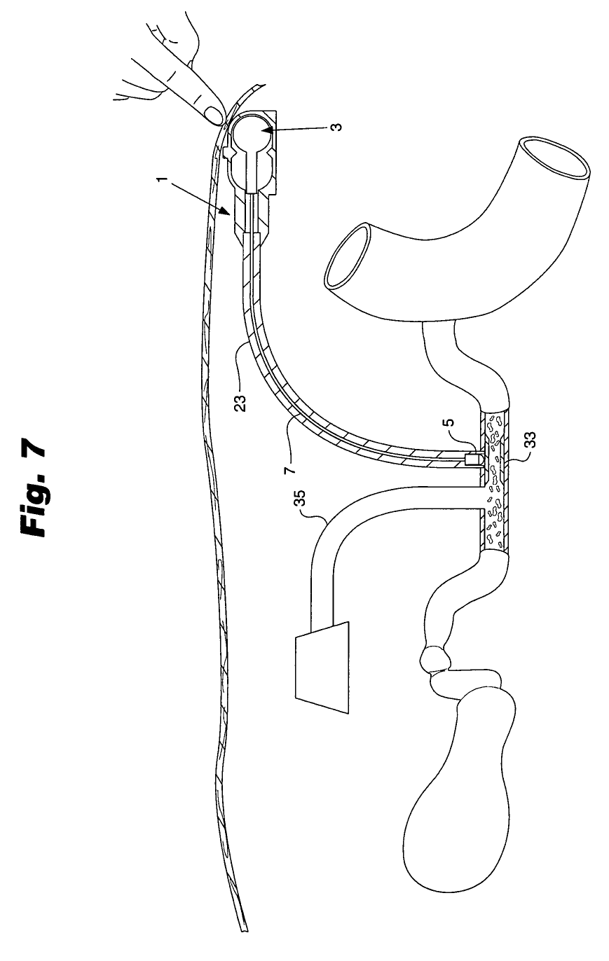

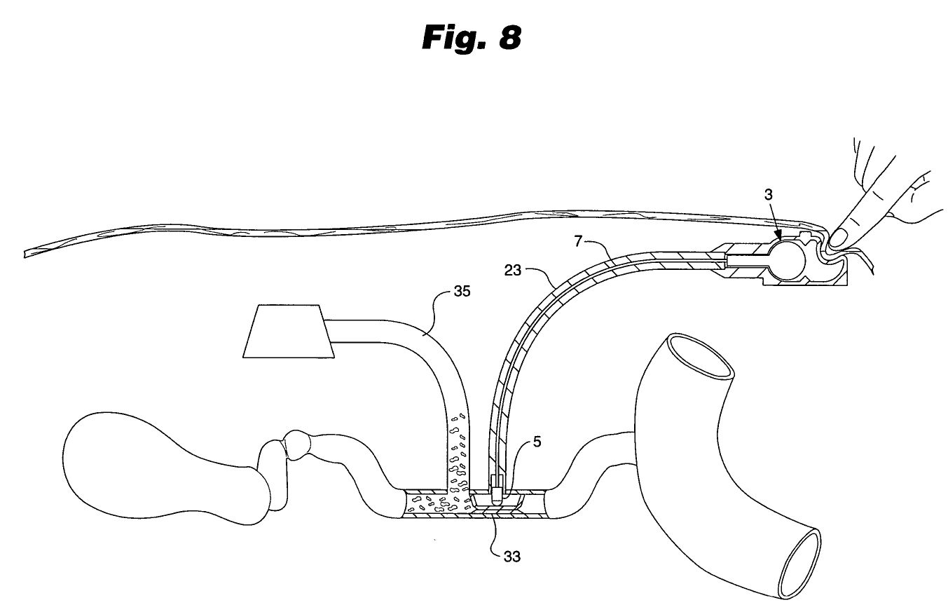

FIGS. 7 and 8 illustrate a system for total bile collection wherein the palpable, implantable actuator designed in accordance with the embodiment as shown in FIGS. 2(a) and (b) of the present invention is used to occlude the bile tube 33 in order to divert flow into the sampling tube 35. FIG. 7 illustrates the user applying force to the palpable, implantable actuator in the direction of the activation means 5 to cause the mechanical switch 3 to move into an “activated” position thereby forcing the connector 7 to extend forward and consequently causing the activation means 5, in FIG. 7 shown as a plug, to extend forward to occlude the bile tube 33. FIG. 8 illustrates the mechanical switch 3 and activation means 5 of the palpable, implantable actuator in the “activated” position wherein the activation means 5 occlude the bile tube 33 and divert the bile to flow into the sampling tube 35.

In one embodiment of the invention, the connector may be a string, thread or suture that connects the mechanical switch to the activation means. In the embodiments illustrated in FIGS. 1(a)-(b), 2(a)-(b), 3(a)-(c), 4(a)-(c), the connector is rigid and possesses column strength in order to extend away from the flexible sheath. In this embodiment, the connector is a string, thread or suture and possesses no column strength. The activation means may be a noose or stirrup used to occlude a vessel or passageway. To cause the palpable, implantable actuator to move into the “activated” position, the user applies force to the palpable portion of the mechanical switch in the direction away from the activation means. In the “activated” position, the mechanical switch pulls the string, thread or suture causing the string, thread or suture to be in tension. The pull and tension in the string, thread or suture causes the activation means to narrow and tighten around the vessel or passageway thereby occluding the vessel or passageway. To cause the palpable, implantable actuator to move into the “deactivated” position, the user applies force to the palpable portion of the mechanical switch in the direction toward the activation means. In the “deactivated” position, the string, thread or suture is no longer in tension and the occluded vessel or passageway becomes unoccluded due to the fluid or pneumatic pressure present in the vessel or the spring force present in the walls of the vessel or passageway.

The palpable, implantable actuator may be used to partially or totally compress the outer wall of any biological or synthetic passageway or organ in order to partially or totally occlude passage of fluid or other material through the lumen of the biological or synthetic passageway or organ. Applications for the palpable, implantable actuator may include occlusion of blood flow through a blood vessel, urinary flow through a urethra, or ingesta through the digestive system.

Other alternatives for activation means include a syringe for the dispensing of medicine, saline or any other liquid; a wire or brush that can clear the lumen of a clogged catheter, vessel or passageway; a noose to strip occlusive clotted blood from the exterior of a catheter; and a tissue expander. The activation means also may be used to activate an implanted medical device. Such activation may include switching on or off or adjusting up or down the power source of an implanted medical device. Notwithstanding the foregoing, the activation means may be any mechanism, component or device used to actuate surrounding bodily tissue, to intervene in or monitor the body's activities, or to actuate an implanted medical device which will in turn actuate surrounding bodily tissue or intervene in or monitor the body's activities.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A palpable, implantable actuator implanted completely within an animal's or human's body to remotely actuate a mechanism, component or device that has been implanted completely within an animal's or human's body and is located distally from the palpable, implantable actuator comprising:
a mechanical switch that is palpable and able to be manipulated through the skin of the body; and
a connector connected to the mechanical switch at one end and at the opposite end to a mechanism, component, device or surrounding bodily tissue or body part which when the mechanical switch is palpated causes the connector to effect a force on such mechanism, component, device or surrounding bodily tissue.

2. A palpable, implantable actuator of claim 1, wherein the mechanical switch is enclosed within a flexible shell.

3. A palpable, implantable actuator of claim 2, wherein said flexible shell has at least one ridge in the interior portion of the flexible shell to secure the mechanical switch in a position.

4. A palpable, implantable actuator of claim 1, wherein the mechanical switch is inverted where the “deactivated” and “activated” positions are reversed.

5. A palpable, implantable actuator of claim 1, wherein the mechanical switch is a toggle switch.

6. A palpable, implantable actuator of claim 5, wherein said toggle switch is spring loaded to prevent inadvertent changing of positions.

7. A palpable, implantable actuator of claim 1, wherein the mechanical switch is a rocker switch.

8. A palpable, implantable actuator of claim 7, wherein said rocker switch is spring loaded to prevent inadvertent changing of positions.

9. A palpable, implantable actuator of claim 1, wherein the mechanical switch is a two position switch.

10. A palpable, implantable actuator of claim 1, wherein the mechanical switch is a multi-position switch.

11. A palpable, implantable actuator of claim 1, wherein there is a sheath that is concentric to the connector and provides a protective barrier and passageway for the connector to pass.

12. A palpable, implantable actuator implanted completely within an animal's or human's body to remotely actuate a mechanism, component or device that has been implanted completely within an animal's or human's body and is located distally from the palpable, implantable actuator comprising:
a mechanical switch that is palpable and able to be manipulated through the skin of the body;
a connector connected to the mechanical switch at one end and at the opposite end to activation means; and
activation means, which connect to the connector, for actuating surrounding bodily tissue, intervening in or monitoring the body's activities, or actuating a device that itself actuates surrounding bodily tissue or intervenes in or monitors the body's activities.

13. A palpable, implantable actuator of claim 12, wherein the mechanical switch is enclosed within a flexible shell.

14. A palpable, implantable actuator of claim 13, wherein said flexible shell has at least one ridge in the interior portion of the flexible shell to secure the mechanical switch in a position.

15. A palpable, implantable actuator of claim 12, wherein the mechanical switch is inverted where the “deactivated” and “activated” positions are reversed.

16. A palpable, implantable actuator of claim 12, wherein the mechanical switch is a toggle switch.

17. A palpable, implantable actuator of claim 16, wherein said toggle switch is spring loaded to prevent inadvertent changing of positions.

18. A palpable, implantable actuator of claim 12, wherein the mechanical switch is a rocker switch.

19. A palpable, implantable actuator of claim 18, wherein said rocker switch is spring loaded to prevent inadvertent changing of positions.

20. A palpable, implantable actuator of claim 12, wherein the mechanical switch is a two position switch.

21. A palpable, implantable actuator of claim 12, wherein the mechanical switch is a multi-position switch.

22. A palpable, implantable actuator of claim 12, wherein there is a sheath that is concentric to the connector and provides a protective barrier and passageway for the connector to pass.

23. A palpable, implantable actuator implanted completely within an animal's or human's body to remotely actuate a mechanism, component or device that has been implanted completely within an animal's or human's body and is located distally from the palpable, implantable actuator comprising:
a mechanical switch that is palpable and able to be manipulated through the skin of the body;
a piston and piston housing that are connected to the mechanical switch at one end and at the opposite end to activation means; and
activation means that connect to the connector for actuating surrounding bodily tissue, intervening in or monitoring the body's activities, or actuating a device that itself actuates surrounding bodily tissue or part or intervenes in or monitors the body's activities.

24. A system for bile collection implanted completely within an animal's or human's body comprising:
a tube for placement in bile duct;
a sampling tube which is connected to and branches off of the bile tube for sampling bile;
a mechanical switch that is palpable and able to be manipulated through the skin of the body;
a connector connected to the mechanical switch at one end and to the activation means at the opposite end; and
activation means, which connect to the connector, for occluding the tube for placement in bile duct when the mechanical switch is activated.
